(19) United States
(12) Patent Application Publication
(10) Pub. No.: US 2013/0189671 A1
(43) Pub. Date: Jul. 25, 2013

(54) Cell Culture System and Method of Use Thereof

(71) Applicants: Jan H. Hoh, Baltimore, MD (US); Jeffrey L. Werbin, Cambridge, MA (US); William F. Heintz, Sykesville, MD (US)

(72) Inventors: Jan H. Hoh, Baltimore, MD (US); Jeffrey L. Werbin, Cambridge, MA (US); William F. Heintz, Sykesville, MD (US)

(21) Appl. No.: 13/816,044

(22) PCT Filed: Aug. 19, 2011
(86) PCT No.: PCT/US11/48513

§ 371 Date: Apr. 12, 2013

Related U.S. Application Data
(60) Provisional application No. 61/375,065, filed on Aug. 19, 2010.

(51) Int. Cl. C12M 1/00

(52) U.S. Cl. CPC C12M 23/34
USPC 435/3; 435/297.1; 435/286.1; 435/288.7; 435/287.1; 435/401; 435/29

(57) ABSTRACT

The present invention is based in part on the discovery of significant improvements to cell culture systems and methods in terms of utility and range of application. The system allows for culture of cells in one or more defined spaces within a culture chamber defined by one or more spaced opposing surfaces to provide significant advantages over previously described culture methods. The system includes a chamber for receiving a fluid culture medium; a first surface and one or more second surfaces defining one or more spaces in the chamber; and one or more cells constrained within the one or more spaces. The second surface includes at least one opening for passage of a substance into the culture space.

### BACKGROUND OF THE INVENTION

1. Field of the Invention

The present invention relates generally to cell culture, and more particularly to a cell culture system having chambers with restricted diffusive exchange, cultured cells, and methods of use thereof.

2. Background Information

Tissues exchange molecules with the blood via diffusion. The nature of diffusive exchange in the tissues was first elucidated by Krogh in the early part of the 20^{th} century. He defined a cylindrical gradient (the Krogh cylinder) along which molecules which that are in high concentration in the blood decrease in concentration as a function of distance from a capillary, and molecules that in high concentration in the tissues increase along the same axis. He found that in general, diffusive exchange in a tissue is limited to a few hundred micrometers beyond which the rate of exchange is insufficient to sustain cells. Thus the gradients that develop by diffusive exchange play a fundamental role in normal organ and tissue function, and restriction of diffusion plays a central role in a number of pathological conditions.

One well-known effect of diffusive exchange in cancer is to constrain the size of tumors to diameters of 300-500 μm absent interior vascularization. While the full complement of relevant molecules whose exchange is important in these microtumors is not known, both oxygen and glucose have received special attention. The role of oxygen deprivation on cancer cells has been widely studied in vitro using hypoxia chambers, in which the partial pressure of oxygen is precisely controlled. It is also straightforward to examine the role of a molecule such as glucose by varying its concentration in culture. However, these types of studies have two significant limitations. First, they fail to account for varying concentration of molecules in the tumors as a function of distance from the nearest blood vessel. In a real tumor, cells along the axis of diffusion experience different concentrations of molecules than their immediate neighbors. Second, manipulating individual molecules leaves open the question of what roles other molecules or combinations of molecules play.

The complex nature of molecular exchange in tumors was appreciated early on, and it was one of the justifications for using multicellular spheroids as tumor models. Spheroids are effectively mini-tumors typically grown in soft agar, and they are formed from isolated cells in culture. Cells are seeded at low density into a soft nutrient matrix, and individual cells nucleate the formation of a group of cells that grows into a spheroidal structure that is typically several hundred micrometers diameter. This overcomes the basic limitation of the molecular manipulation approach and improves experimental access relative to microtumors in vivo. However, high-resolution optical imaging of spheroids is challenging—particularly in significant numbers. Although some improvement is obtained using the cylindroid approach in which spheroids are flattened to improve imaging. Further, the same properties that make the spheroid system so important can make manipulation of the system difficult.

Limitations imposed by diffusive exchange also play a role in capillary density in the tissues. Diffusive exchange also plays an important role in any pathological condition in which blood flow to a tissue is reduced or abrogated in some fashion, such as heart attacks, strokes and renal ischemia. Further, the diffusive exchange is also the primary mechanism by which many therapeutic agents (e.g., drugs) are delivered to the tissue, and as a result the concentration of such agents is non-uniform in the tissues. Toxic agents can also be distributed to the tissues via diffusion from capillaries or other blood vessels.

There are a limited number of other approaches and models to studying this problem ex vivo. As noted above, it is common to manipulate the concentrations of various molecules that cells are exposed to in culture—however these manipulations fail to reproduce the gradients that are characteristic of diffusive exchange. One approach that does reproduce the diffusive gradients is the so-called Sandwich System initially proposed by Hlatky and Alpen (1985). Briefly, the Sandwich System is composed of two glass slides separated by a spacer (60-300 μm), and cells are seeded into the gap between the slides. The slides are cultured such that there is free exchange with bulk media at the edge of the slides (FIG. 1).

FIG. 1 is a schematic of the sandwich system for 2D restricted diffusion developed by Hlatky and Alpen. FIG. 1A depicts two sandwiches in top view. FIG. 1B depicts the edge of one sandwich in cross section. The system is composed of a pair of slides stacked on top of each other, such that their xy positions overlap—and separated in z by a spacer. Cells are seeded into the gap between the slides from the long side. As the cells grow and metabolize materials, the small gap between the slides limits the exchange between the bulk medium (surrounding the sandwiches) and the center of slides. This results in a gradient of oxygen and other molecules at the edge of the sandwich (x_{b}).

The sandwich geometry has the effect of establishing a gradient between the edge of the slides and the inside. As cells grow in the slides, the cells nearest the edge tend to consume more the nutrients—restricting growth of cells further from the edge. The Sandwich System therefore provides gradients of all molecules that are exchanged from the inside of the gap with the outside, while also permitting optical microscopy and access to experimental manipulation.

The Sandwich System has since been used to study for example the interplay between that oxygen and glucose deprivation in giving rise to necrosis and spatial differences in tumor sensitivity to x-rays. More recently Jain and coworkers have implemented a version of the sandwich model, and shown that vascular endothelial cells in a sandwich spontaneously rearrange into vessel like networks. They have also modeled the effect of a sandwich environment on stem cell development. But in the years since its development, the sandwich model has changed little.

Gradients of molecules in cell culture can also be established by using a narrow channel, containing cells, to separate two reservoirs in which there is one or more molecules of different concentrations (in relatively large volumes). The molecules diffuse through the channel, establishing a gradient along the channel in which the cells are growing. The small dimension of the channel in these types of devices is often on the order of 100 μm. These devices are frequently used to analyze chemotaxis in eukaryotic cells.

Gradients of molecules for cell culture can also be established using microfluidic systems. Here the flow of a solution is mixed with another solution in which the concentration of one or more molecular components varies, such that the concentration of the molecule(s) of interest varies as a function of time or space.

Confinement has also been employed in measuring the metabolic rates of cells. In one such system, cells are first grown in a dish or a microtiter plate. A plunger is then brought into close proximity (or soft contact) to one or more cells, forming a narrow gap between the surface of the plunger and the surface on which the cells are growing. The size of the gap limits the diffusive exchange with the medium, and thus causing an excess of metabolic products to accumulate near the cells. Sensors in the system then report changes in pH or other metabolic products of interest. The change of these products as a function of time is used to quantify metabolic rates.

### SUMMARY OF THE INVENTION

The present invention is based in part on the discovery of significant improvements to cell culture systems and methods in terms of utility and range of application. The system allows for culture of cells in a defined space with a culture chamber defined by spaced opposing surfaces, and an opening in one surface through which diffusion can occur, to provide significant advantages over previously described culture methods. Using a space with a defined shape and an opening with a defined shape allows for precise control the environment of each cell. This can be used to mimic a biological niche or create many other environments of interest.

Accordingly, in one aspect, the present invention provides a cell culture system for culturing cells. The system includes a chamber for receiving a fluid culture medium; a first surface and one or more second surfaces defining one or more spaces in the chamber; and one or more cells constrained within the one or more spaces. The first and one or more second surfaces are in opposition and separated by one or more spacers or otherwise separated to define one or more culture spaces. The second surfaces include at least one opening for passage of a substance into the culture space. The culture system may further include a fluid culture medium, for example within the chamber space and/or in fluid communication with the chamber space, for example through the opening. A gradient of one or more substances in the culture medium as a function of distance from the opening develops. The form of the gradient is related to the shape of the culture space and the shape, position and number of openings.

In another aspect, the present invention provides a method of cell culture utilizing the system described herein. The method includes a) providing a chamber having a first surface in opposition to one or more second surfaces defining one or more spaces in the chamber, the first surface and second surfaces being spaced at a distance to allow one or more cells to adhere to the first surface or grow within the space; b) introducing one or more cells to the one or more spaces in the chamber, wherein the one or more spaces comprise a fluid culture medium; and c) providing to the cell a substance, wherein the substance is provided through one or more openings in one or more second surfaces, wherein a concentration gradient of the substance within the one or more spaces is established, thereby culturing the cells. In various embodiments, cells are allowed to adhere to one of the surfaces and are cultured between the first surface and the second surface. In some embodiments, the cells may be cultured as a monolayer of cells. In various embodiments, multiple substances may be provided during culture through one opening, or additional openings to establish additional concentration gradients of multiple substances within the space. In some embodiments, the method further includes obtaining an image of the cells, via visual inspection or using an imaging device. In some embodiments, the method may further include detecting one or more of molecular concentration of a substance or physical parameter of the culture system or contents thereof.

In another aspect, the present invention provides a method for assaying healing or recovery responses of cells using the culture system and method described herein.

In another aspect, the present invention provides a method for assaying healing or recovery responses of cells. In various embodiments, cultured cells may be monitored before and after specific growth conditions or exposure to agents. For example, cells may be culture using the culture system according to the method described herein, wherein the second surface is removed and the cells are further cultured without the second surface being present. The growth of the cells before and after removal of the second surfaces may be compared to assess various parameters of the culture conditions.

In another aspect, the culture system and method may be adapted for modeling studying tumors.

In another aspect, the culture system and method may be adapted for modeling and studying ischemia.

In another aspect, the present invention provides a method for performing a cell migration assay using the culture system and method described herein.

In another aspect, the present invention provides a method for performing a cell migration assay. The method includes a) providing a chamber having a first surface in opposition to one or more second surfaces defining one or more spaces in the chamber, the first surface and second surfaces being spaced at a distance to allow to be cultured between the first surface and the second surfaces; b) introducing one or more cells to the one or more spaces in the chamber, wherein the spaces comprise a fluid culture medium; c) providing to the cell a substance, wherein the substance is provided through an one or more openings in the second surfaces, wherein a concentration gradient of the substance within the one or more spaces is established; and d) detecting an increase or decrease in migration of the one or more cells along the concentration gradient, thereby performing a cell migration assay.

In another aspect, the present invention provides a method of performing a titration assay using the culture system and method described herein.

In another aspect, the present invention provides a method of performing a titration assay. The method includes a) providing a chamber having a first surface in opposition to one or more second surfaces defining one or more spaces in the chamber, the first surface and second surfaces being spaced at a distance to allow culture of one or more cells between the first surface and the second surfaces; b) introducing one or more cells to the spaces in the chamber, wherein the spaces comprise a fluid culture medium; c) providing a substance to the one or more spaces and allowing at least one concentration gradient of the substance within the one or more spaces to be established via one or more openings in the one or more second surfaces; and d) detecting a response of the one or more cells to the substance at time intervals as the concentration gradient is established, or detecting a response at different positions along the gradient, thereby performing a titration assay.

In another aspect, the present invention provides a method of generating a tissue or tissue mimic using the culture system and method described herein.

In another aspect, the present invention provides a method of generating a tissue or tissue mimic. The method includes a) providing one or more cells of a type appropriate for a specific tissue type; b) culturing the cells using the system and method described herein; and c) harvesting the resultant tissue, thereby generating a tissue. The differentiation and growth of the cells may be controlled via introduction and selection of the substance in combination with configuration of the system. The various embodiments, the tissue may be a one, two or three-dimensional tissue, and incorporate one, or a variety of specialized cell types.

### BRIEF DESCRIPTION OF THE DRAWINGS

FIGS. 1A-1B are top and side views, respectively, of a prior art cell culture system.

FIG. 2A is a tope and cross-sectional side view of a cell culture system in an embodiment of the invention.

FIG. 2B is a top and side view of a coverslip top in an embodiment of a culture system according to the present invention.

FIG. 2C is a top and side view of a coverslip top in an embodiment of a culture system according to the present invention.

FIG. 2D is a schematic illustrating cell culture using the coverslips depicted in FIGS. 2B-2C.

FIG. 3A is a top view of a 20 mm diameter circular coverslip having a microdrilled opening of 200 μm in diameter in an embodiment of a culture system according to the present invention.

FIG. 3B is a top view of a 20 mm diameter circular coverslip having a microdrilled bar shaped opening of 400 μm wide in an embodiment of a culture system according to the present invention.

FIG. 4 is a graph plotting data pertaining to partial pressures of oxygen obtainable using a cell culture system in an embodiment of a culture system according to the present invention.

FIG. 5 is a schematic showing dimensional reduction from a three dimensional spheroid to a one dimensional geometry representative of the three dimensional spheroid for positioning opening in a coverslip in an embodiment according to the present invention.

FIG. 6 is a graph plotting data pertaining to relative density of cells grown in a culture chamber having two bar shaped openings representative of the one dimensional tumor model geometry.

FIGS. 7A-7R are top views of circular coverslips in various embodiments according to the present invention.

FIGS. 8A-8I are top views of rectangular coverslips in various embodiments according to the present invention.

FIGS. 9A-9J are cross-sectional side views of culture chambers in various embodiments according to the present invention.

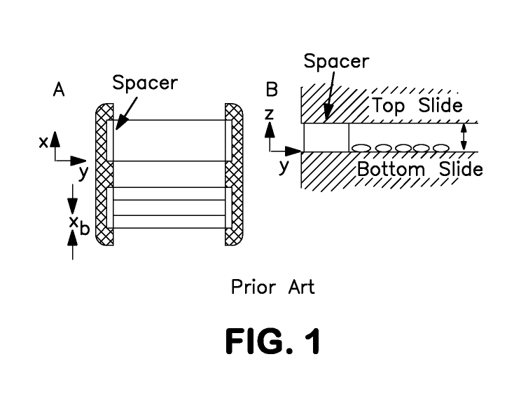

### DETAILED DESCRIPTION OF THE INVENTION

The present invention is based in part on the discovery of significant improvements to cell culture systems and methods in terms of utility and range of application. The system allows for culture of cells in a define space with a culture chamber defined by spaced opposing surfaces and an opening in a surface to provide significant advantages over previously described culture methods. Using a defined space allows for precise control the environment of each cell allowing for culture of cells in an environment that mimics a biological niche.

The present invention provides a cell culture system that allows culture of cells in vitro in a microenvironment that may be finely controlled. Control of a cell's microenvironment allows for the cell to be cultured in an environment that mimics an in vivo biological environment. Additionally, controlling the microenvironments allows for various assays to be performed by taking advantage of various aspects of the cell culture system.

The cell culture system of the present invention provides a series of advantages over existing culture systems. The culture system of the present invention allows cells to experience gradients of substances, for example, culture substances or test substances, in the fluid culture medium. Gradients may be generated or maintained by biochemical or physiological processes in the cells, such as synthesis of new substances, degradation of existing substances, binding of a substance or release of a substance. Gradients may also be generated or maintained by a sink or a source of substances, such as a small bead with antibodies to bind a substance or a bead impregnated with a substance to release said substance.

The culture system of the present invention also allows cells to experience self-generated gradients of all molecules that are consumed or secreted for short or extended periods of time. The system is compatible with standard cell cultureware, including multiwell plates, and therefore can be used in high content screening or other high throughput systems. The system also facilitates observation and imaging of cells since the chamber covers may be easily removed from and optionally put back at a later time, thus allowing examination of cellular processes (such as matrix remodeling) and their recovery during and after acute restriction of exchange. One skilled in the art would understand that culturing cells between two planar substrates facilitates direct observation of cell growth and subcellular detail in live cells with imaging techniques such as standard bright field imaging or fluorescent imaging without the use of a confocal microscope or a laser imaging device, although confocal microscopy and laser-based imaging devices may also be used to observe live cells in this system. When cells are confined within the proximity of the in-focus-plane of an objective lens, the image quality can significantly improve as compared to images of cells that can move away from the in-focus-plane.

Further, the form of the gradients to which cells are exposed can be manipulated by providing different shapes of openings, or positioning the openings in different ways in association with the culture chamber. This for example allows control over the form of gradients within the chamber and in the cellular microenvironment. Even further, data analysis can be simplified and automated by the design of the openings in association with the culture chamber.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an” and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

The present invention provides a cell culture system for culturing cells. The system includes a chamber for receiving a fluid culture medium; a first surface and one or more second surfaces defining one or more spaces within the chamber; and one or more cells constrained within the one or more spaces. The one or more second surfaces include at least one opening for passage of a substance into the one or more spaces.

As used herein, the term “restricted exchange environment chamber” or “REEC” refers to the culture system of the invention and used synonymously therewith.

In another aspect, the present invention provides a method of cell culture utilizing the system described herein. The method includes a) providing a chamber having a first surface in opposition to one or more second surfaces defining one or more spaces in the chamber, the first surface and one or more second surfaces being spaced at a distance to allow one or more cells to adhere to the first surface or grow within the spaces; b) introducing one or more cells to the one or more spaces in the chamber, wherein the one or more spaces may include a fluid culture medium; and c) providing to the cells a substance, wherein the substance is provided through one or more openings in the one or more second surfaces, wherein a concentration gradient of the substance within the space is established, thereby culturing the cells.

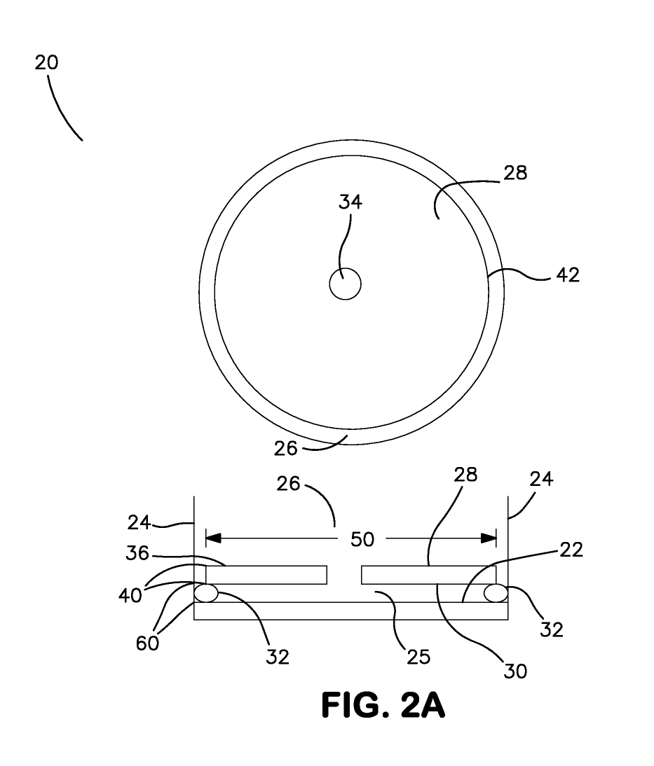

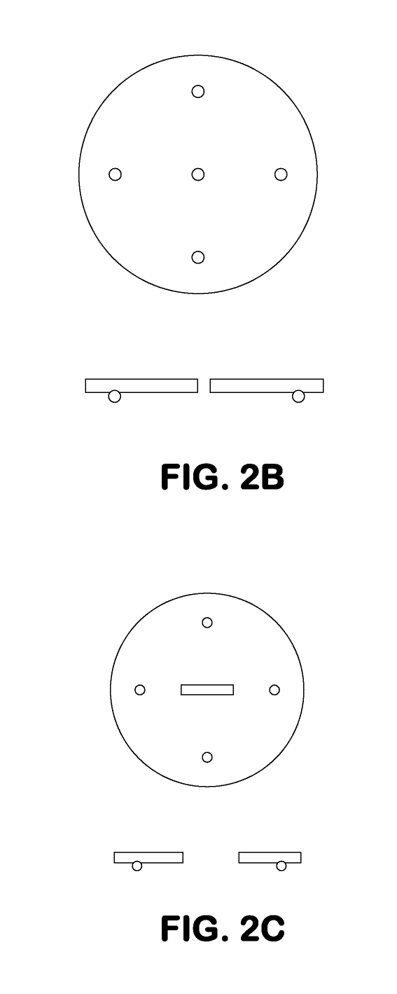

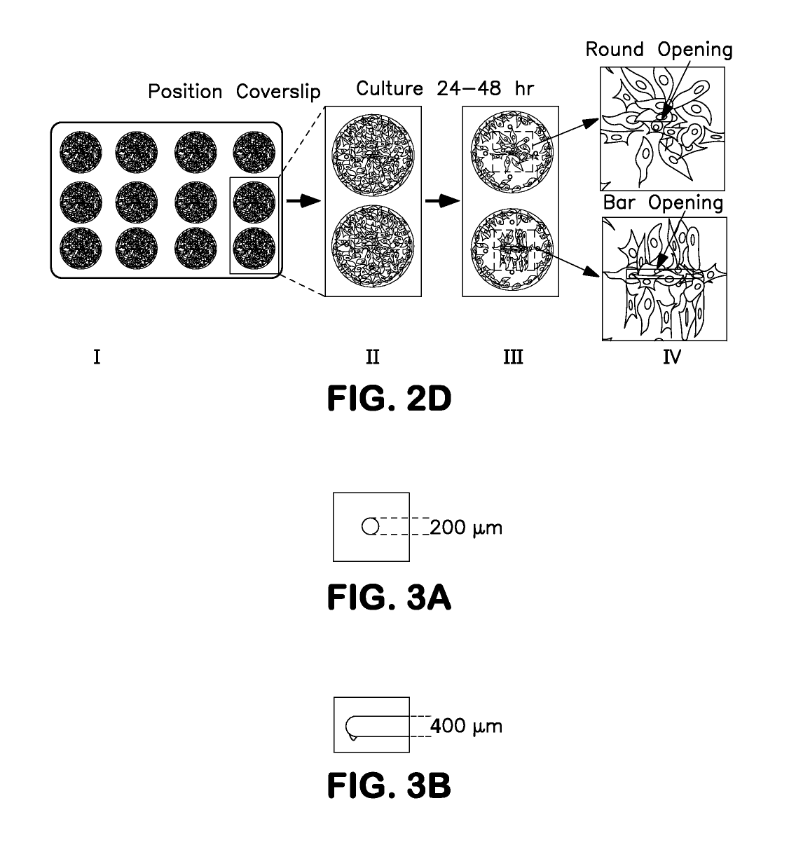

Referring to FIGS. 2A-2D, an illustrative embodiment of the invention includes a cell culturing system 20. System 20 has a base or first surface 22 and side walls 24 formed generally perpendicular to the base surface 22, thus forming a chamber 26 for receiving and containing a liquid culture medium. System 20 further includes a cover or plate 28 defining a second surface 30 in opposition to the base surface 22, the surfaces being spaced at a distance defined by spacers 32. The first surface 22 and the second surface 30 define a space or volume 25 within the chamber 26 for culturing of cells. In this embodiment, the cover 28 is positioned over base surface 22 inside chamber 26 and is loosely and removably fitted within the chamber.

One of skill in the art would understand that base surface 22 and second surface 30 may be shaped in a variety of ways to define the particular culture space. For example either or both surfaces may be generally planar, curved, angled, or be of a geometry that includes any combination of planar, curved or angled regions.

As is described further below, base surface 22 and second surface 30 form barriers between which cells are constrained and cultured within a controllable microenvironment. The cover or plate 28 includes at least one opening 34 to allow for passage of a substance.

FIGS. 2B and 2C are top views and cross-sectional views of two configurations of covers 28 in embodiments of the restricted exchange environment chambers of the present invention. The top views show round covers 28, with two different opening 34 configurations. The cover 28 shown in FIG. 2B has a circular opening 34 which passes through the width or thickness of cover 28. The cover 28 shown in FIG. 2C has a bar shaped opening 34 which passes through the width or thickness of cover 28. Spacers 32 are spheres attached to the bottom of cover 28 (dimensions are not to scale). Spacers 32 and openings 34 are made larger for clarity.

FIGS. 3A and 3B are partial top views of the cover configurations depicted in FIGS. 2A and 2B in which the openings have specific dimensions and the covers are constructed of specific materials. FIG. 3A shows a glass cover 28 having an opening 34 that is a 200 μm hole through the thickness of cover 28, while FIG. 3C shows a glass cover 28 having a bar shaped opening 34 having a width of 400 μm.

FIG. 2D is a schematic illustrating cell culture using the covers 28 shown in FIGS. 2B-2C and chamber 26 of FIG. 2A with the system being adapted to a multiwell plate format including multiple chambers 28 (shown in panel I). In this embodiment, cells are first introduced into chamber 28 and grown to desired confluency, after which covers 28, as shown in FIGS. 2A and 2B, are placed in the wells. The cells are then cultured for an additional time, for example 24-48 hours, during which time various gradients are established in space 25 and cells in each chamber 28 adjust their morphology and metabolism. This may include cell death or apoptosis in certain parts of the chamber. Panel IV shows a high magnification schematic of one possible response of cells to two different opening geometries.

Using the REEC as an illustrative embodiment, the REEC is a culture system having a culture chamber with the following characteristics: 1) a base or first surface 22 upon which cells may be grown; 2) a cover 28 defining a second surface 30 interior to the chamber 26 separated by a distance defined by spacers 32; a space 25 defined by the volume between the first surface 22 and the second surface 30; a cover 28 having a thickness 40, a perimeter edge 42, interior surface 30 generally opposing first surface 22, exterior surface 36; one or more openings 34 in cover 28 allowing for transfer of at least one substance across the cover 28.

The REECs of the present invention include a culture space or volume 25 within chamber 26 defined by a first surface 22 and a second surface 30. In various embodiments, culture space 25 is defined by a major dimension 50 and a minor dimension 60, the minor dimension being defined by the distance between first surface 22 and second surface 30. The minor dimension 60 is generally small compared to the major dimension 50. In various embodiments, the major dimension 50 is at least about 2, 3, 5, 10, 20, 50 or 100 times larger than the minor dimension 60. Minor dimension 60 may be less than about 5, 2, 1, 0.5, 0.1, or 0.05 mm. In some embodiments, the minor dimension 60 may the thickness of a single cell.

In some embodiments, the distance between the first 22 and second 30 surfaces is comparable to the size of a single cell to be cultured, such that the cells grow as a monolayer. In such an embodiment, it will be appreciated that a cultured cell would touch both first 22 and second surfaces 30. The distance may vary depending on the size of the cells to be cultured. In various embodiments, the distance between first 22 and second 30 surfaces may be about 0.05 to 250 microns or about 2 to 40 microns.

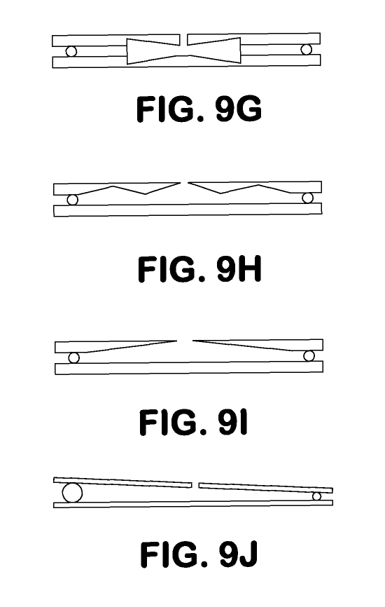

In some embodiments the minor dimension is uniform, such as depicted in FIG. 9E.

In some embodiments the minor dimension is not uniform, such as depicted in FIG. 9J.

As would be understood by one skilled in the art, restricted exchange environment chambers of the present invention may be implemented in many equivalent ways. As would be recognized by one of skill in the art, cover 28 may be held in position in a variety of ways, such as by a hinge, a weight, a lid, a clip or other interlocking members disposed on cover 28 and another surface of the chamber. In one embodiment cover 28 is loosely fitted within chamber 26. In another embodiment, cover 28 is held in place via an adhesive material, such as silicone adhesive or other inert, biocompatible adhesive. Alternatively, cover 28 may be fixed by other chemical or mechanical holding mechanisms. Cover 28 may be removably mounted by a hinge on base plate 22.

In various embodiments, first surface 22 and second surface 30 are spaced apart to define the minor dimension of the culture space 25. In some embodiments spacers 32 are provided between the surfaces before, during, or after cells are loaded. Suitable spacers are known in the art and include by way of illustration, glass spheres, thin membranes, gaskets, small pieces of polystyrene (e.g., stubs that are part of the surface in which the cells are growing), material machined from the plate, polymer, and the like. In some embodiments, the spacers are attached to or are part of the cover 28, e.g. spacers molded into a polystyrene cover 28. In some embodiments, the spacers can be fluorescent, optionally coated with biomolecules or reactive chemical functional groups or magnetic. In some embodiments, the spacers are rigid and resist compression to prevent cells from being overly compressed. In some embodiments, the spacers are of uniform size. In other embodiments, spacers are of different sizes. When the spacers are different, a wedge shaped space 25 may be formed (FIG. 9J).

In alternative embodiments, separation of the surfaces may be achieved without the use of spacers. The separation of the surfaces may be achieved by holding two surfaces 22 and 30 apart. For example, in a multiwell plate, cover 28 may be mounted on the sidewalls of a well defining chamber 26 or held by an insert that is mounted to the top of the well. In other embodiments, the relative positions of the surfaces may be changed at one or more times during the culturing of cells resulting in different separations of surfaces as a function of time.

Cells are incubated in the REECs of the present invention for some desired period of time during which time the dynamics of cells in response to the substance gradients established via openings 34 may be monitored or recorded, optically or otherwise as discussed below. Cover 28 may be removed, and the cells in the chamber fixed to preserve the sample for later analysis with antibodies or other reagents. In one embodiment, an additional substrate is placed in the bottom of the chamber prior to plating cells. The additional substrate may be recovered for higher performance microscopy than would be possible through a transparent base plate.

As discussed further below, the cells cultured within the REEC are exposed to concentration gradients of substances, for example, but in not limited to, molecules comprising the fluid culture medium, molecules that are consumed and secreted by cells, or test substances—molecules added to the fluid culture medium, as a function of distance from the point of the chamber space 25 in contact with the bulk medium. Thus it can be expected for an REEC with a round opening for the cells in the center of the chamber space 25 below the opening 34 to grow normally, while at increasing distances from the opening 34, cells in chamber space 25 will experience conditions similar to those in the center of spheroids or microtumors that restrict growth.

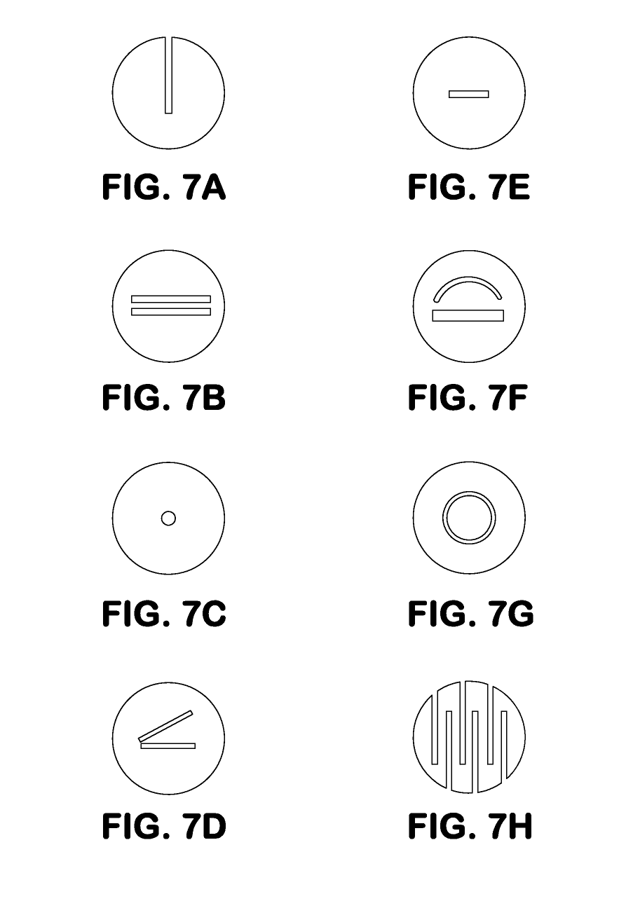

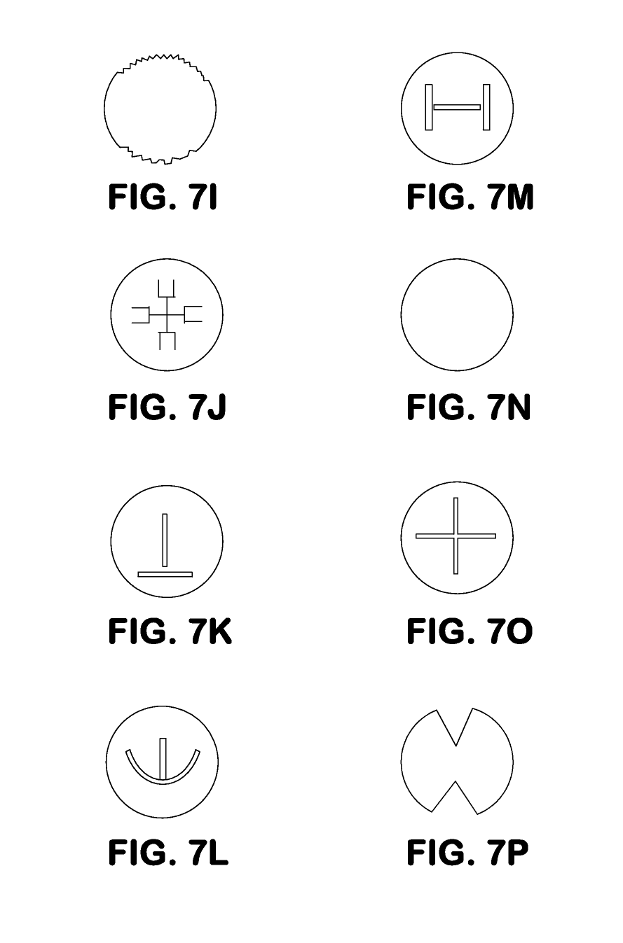

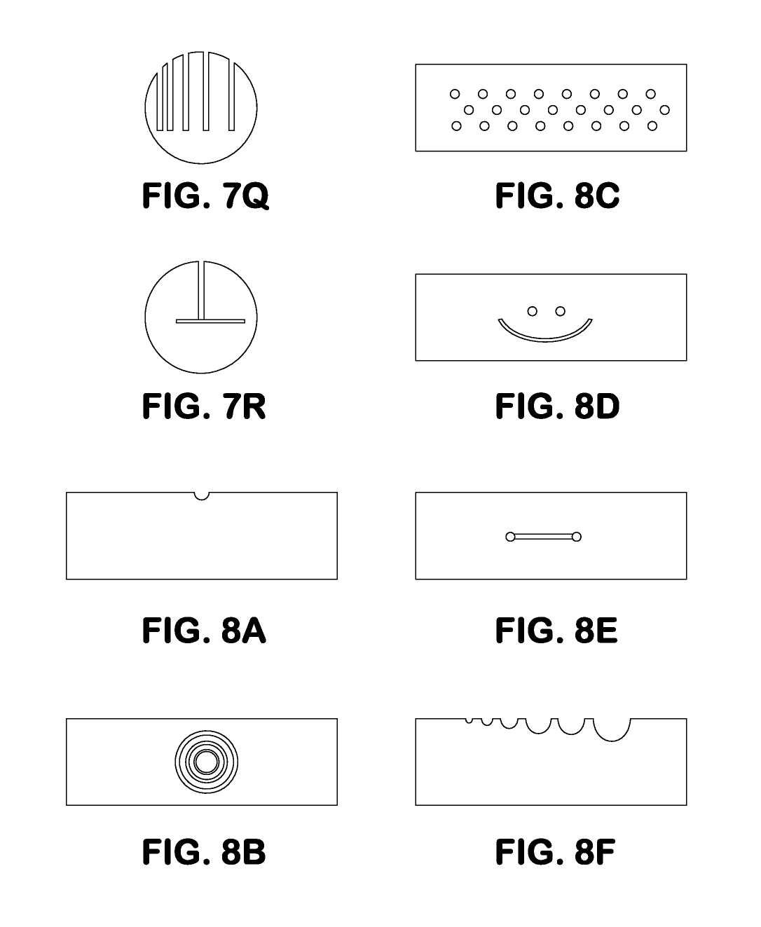

The shape and position of openings 34 can be controlled in a way as to produce gradients with specific types of shapes or properties. Some examples are shown in FIGS. 7-9. Likewise, shape of the edges of an REEC can be controlled to produce gradients with specific shapes or properties.

As discussed above, base surface 22 and second surface 30 may be shaped in a variety of ways to define the particular culture space. For example either or both surfaces may be generally planar, curved, angled, or be of a geometry that includes any combination of planar, curved or angled regions. As such, the thickness of the cover 28 and the base plate defining the base or first surface 22, may vary with position. FIGS. 9E-9H show REEC embodiments in which the thickness of either or both the cover 28 and the base plate 22 vary with position.

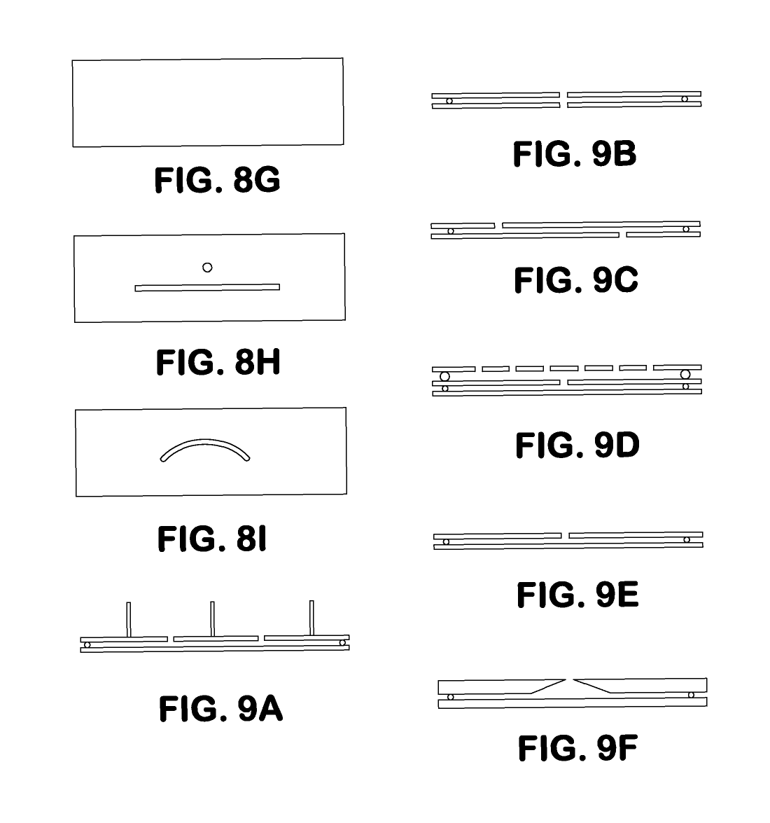

In various embodiments, cover 28 may be a variety of different shapes. For example cover 28 may be elliptical, circular, rectangular, square, or virtually any desired regular or irregular geometric shape. FIGS. 7A-7R show a variety of circular covers 28, while FIGS. 8A-8I show a variety of rectangular covers 28. The cover may be sized for use with any commercially available culture plate, including single well and multiwell plates, or any other commercially available cultureware, including Petri dishes, or other chambers.

In various embodiments, cover 28 maybe a microscope slide, coverslip or other thin plate. In general the materials suitable for constructing the REECs are well known to a person skilled in the art and include any material that is compatible with cell culture.

The base plate 22 and cover 28 may be made of the same or different materials. Suitable materials include (porous) glass, quartz, plastic, resin, laminates, silicon, polymer, polymer membrane, polydimethylsiloxane, glass polystyrene matrix or polyethylene, natural or synthetic biocompatible polymers, or other biocompatible materials known in the art. The base plate 22 and cover 28 may be made of untreated, bio-inert glass or untreated polystyrene or polycarbonate, or may be treated or include surface modifications as discussed further below. The base plate 22 and cover 28 may be porous or gelatinous, such as a hydrogel, a filtration membrane or dialysis membrane.

The material used to construct an REEC system of the present invention may be opaque or translucent. In some embodiments, translucent or transparent materials are utilized to permit cells to be optically monitored. Transparent or translucent materials can also facilitate the use of light to stimulate certain chemical or biological changes in the culture chamber or in the cells. In various embodiments, base plate 22 and/or cover 28 may be transparent to visible, UV, or IR light. The base plate 22 and cover 28 also be opaque. The base plate 22 and cover 28 may also filter or pass specific wave lengths of light.

One or more of first surface 22 and the second surface 30 may be derivatized or coated before or during culture with extracellular matrix molecules, adhesion ligands, growth factors, receptors, and the like. The use and benefits of coating the inner surfaces of a culture chamber is known to a person skilled in the art.

As can be appreciated, cells can attach to the surfaces they contact, which may or may not be desirable. To limit attachment of cells, first 22 and second 30 surfaces may be made of or lined with substances that limit adhesion, such as untreated polystyrene, glass, polyacrylamide gel, or anti-adhesive biomolecules including polysaccharides, proteoglycans, proteins, or polyethylene oxide. Thus, the cells can be confined in space 25 without necessarily attaching or adhering to the first 22 and second 30 surfaces. For example, one or both of the first 22 and second 30 surfaces may be coated with materials that affect cell attachment and behavior, such as poly(dimethylacrylamide) or dimethyldichlorosilane.

The first 22 and second 30 surfaces may also be modified or coated to increase affinity to a certain cell type to control the cell growth. When the surfaces have no or little affinity to the cultured cells, the cultured cells may be confined but do not attach to the surfaces of the cover plates, and thus they are mobile and may move laterally along the first 22 and second 30 surfaces. For example, cells may move due to a concentration gradient of a substance in the culture medium or they may be forced to move by an external force such as a magnetic force. Cells may orient in a certain way depending on the surface properties. The surface materials may also be so selected as to test compounds that attract or repel a certain cell.

In one embodiment, the first 22 and/or second 30 surfaces may be made, modified or coated with a material specifically selected to either facilitate or inhibit the growth of a certain cell or cell type.

The inner surfaces of the culture space 25 may be made reactive so that other molecules may be covalently linked. The surface can be made reactive in various ways known to those skilled in the art, for example by treatment with such molecules as aminopropyltrimethoxysilane (APTS), which presents amine groups on the surface. Thin layers or bulk materials may be linked to the surface. Bulk materials include gels made from protein, polyacrylamide, or other materials. Such gels may be formed in moulds made with standard microfabrication techniques. The gels may be placed on the surface and covalently bound into place by reaction with the activated surfaces. For example, collagens or fibronectin may be used.

The surfaces may be derivatized with binding proteins that a target cell type is normally exposed to in a natural environment, such as claudin and occludin (for tight junctions), cadherins (for actin-linked, adherens junctions), co unexins (for gap junctions), and selectins (for selectin-lectin interactions)). Thin layers of proteins may be patterned on the inner surfaces of chamber 26, for example by treatment of the APTS-treated surface with glutaraldehyde, or with the photoactivatable cross-linker 4-benzoylbenzoic acid succinimidyl ester, or by using other techniques known to those skilled in the art. The proteins may be of any type. The proteins may be patterned in concentration gradients on niche-bounding surfaces by methods known to those skilled in the art.

The first 22 and second 30 surfaces may be very rigid or somewhat soft and flexible, depending on the cells to be cultured and the intended application. Rigid surfaces can be used to confine cells with precision. Soft barriers can be used in applications where it is desirable to allow cells to expand or grow in size to a limited extend, such as when culturing bone cells, lens cells and other hard tissues. Cover 28 may also be made of shape memory materials or photo-electric shape responsive polymers. Such covers may be used when it is desirable to dynamically vary the distance between barriers during cell culture, such as to periodically vary the compression or tension that a cell experiences.

As will be appreciated by a skilled person, growing cells in a monolayer can be advantageous. For example, it facilitates monitoring of the cells in culture because the optical path across a monolayer can be short; it allows the direct manipulation of neighboring cells to control the target cell's environment (e.g., by placing or removing a neighbouring stromal cell); and it permits the precise positioning of sources and sinks of substances at the target cell.

In various embodiments, at least one opening 34 is provided in the cover 28 of the REEC to allow exchange of at least one substance between the culture space 25 and exterior volume in the exterior side of the cover 28. As used herein, an opening 34 is intended to refer to a feature in the cover 28 where the rate of diffusive exchange of a substance, such as a small molecule, across the thickness 40 of the cover 28 is more rapid as compared to areas of the cover 28 without the feature. One of skill in the art would appreciate that an opening 34 may be positioned at any location in the cover 28, for example fully within the perimeter edge 42, or intersect edge 42 of the cover 28. One of skill in the art would also appreciate that while an opening may be free of all material, an opening need not be free of all material, but rather may be filled with a material that modulates or controls the rate of exchange of one or more substances. In various embodiments, opening 34 is in contact with a bulk solution that is compatible and suitable for the culture of cells in the space 25 within the chamber 26.

An opening of the present invention may be a variety of shapes and sizes. For example, the openings 34 in the embodiment shown in FIG. 2B is circular, while the opening in the embodiment shown in FIG. 2C is rectangular or bar shaped. FIGS. 7A-7R show a variety of circular covers 28 having a variety of configurations of openings 34. FIGS. 8A-8I show a variety of rectangular covers 28, having a variety of configurations of openings 34.

In various embodiments, an opening 34 may have a dimension parallel to the major dimension of cover 28 of less than about 90, 80, 70, 60, 50, 40, 30, 20, 10, 5, or 1% of the major dimension of the second surface 30 defining culture space 25. The opening 34 may have an area parallel to the major dimension of cover 28 of less than about 90, 80, 70, 60, 50, 40, 30, 20, 10, 5, or 1% of the area parallel to the major dimension of the second surface 30 defining culture space 25.

An opening 34 may be made using a variety of techniques known in the art. For example such techniques may include, but are not limited to, machining, drilling, milling, laser machining, punching, die casting, molding, etching, and the like.

Examples of various geometries and configurations of openings are shown in FIGS. 7-9. The embodiments shown include basic primitive geometric configurations (i.e. building blocks) of openings and covers that can be combined singly or multiply into a REEC culture system of the present invention. FIGS. 7-9 contains round and rectangular covers with one or more patterns of one or more openings of one or more different geometries. However, as will be appreciated, the one or more patterns of the one or more openings and their one or more geometries may be made in a cover of any shape, and are therefore not limited to those shown. The one or more geometries of the one or more openings and the position of the one or more openings define the diffusive exchange of one or more substances or small molecules in the system and the gradients of substances or small molecules in the space 25.

Primitive geometric elements include openings of a single round hole (FIG. 7C) with a diameter, a single straight line with a constant length and a constant width (FIG. 7E), a curved line or arc of a circle with a constant width and a constant radius of curvature (FIG. 8I), an opening with a varying cross sectional area as a function of depth through the plate (FIG. 9F and FIG. 9I), the intersection of one of the above openings with the edge of the plate (e.g., FIGS. 7A and 8A), a single line with one or more widths and one or more radii of curvature (FIG. 7N and FIG. 8G), and a plate with a thickness that varies with position (FIG. 9G). An opening may be an open or closed opening. An open opening is an opening with a perimeter that intersects the edge of the second surface (e.g., FIG. 7A). A closed opening is an opening which is wholly interior to the second surface, such that the perimeter of the opening does not intersect the edge of the second surface (e.g., FIG. 7C). Other embodiments including primitive geometric elements which are not pictured include regular and irregular polygonal openings, wedge-shaped openings, and the like.

One or more primitive geometric elements may be combined in a system to generate complex diffusive exchange within the culture chamber. The gradients may be established by the positions, orientations, and distances of openings 34 relative to each other and the edges of the plate, and they can be specified (i.e. tuned or adjusted) depending on the experimental situation. Some examples of one or more openings combined into a system of the present invention include openings of two parallel lines (FIG. 7B), two straight lines at an angle (FIGS. 7D and 7K), a circular hole and a straight line (FIG. 8H), two arcs of equal radius of curvature with convex sides facing each other (FIG. 7G), multiple intersections of primitive elements with the edge of the plate (FIGS. 7H, 7Q, 7I, 7R, 8F), multiple circular openings of identical diameters in a regular array (FIG. 8C), and straight or curved lines in a fractal or snowflake-like pattern (FIG. 7J).

Some of the geometries depicted in FIGS. 7-9 are analogs or models of in vivo geometries and environments or analogs or models of in vitro systems. For example FIG. 8C models the diffusive environment of a Krogh cylinder, in which the capillaries of striated muscle are arranged with a regular spacing dependent on the intensity of the metabolism. The embodiment in FIG. 7B can function as a one-dimensional tumor model as discussed in the examples in which cell growth between the two linear openings are in a diffusive environment similar to that of a three-dimensional tumor. The embodiment in FIG. 7G provides a two-dimensional tumor model. The embodiment in FIG. 7C can be used to examine the responses of cells to a point-source or point sink, or as two-dimensional model for diffusion to and from a capillary.

As can be appreciated, openings 34 allow for a concentration gradient of a substance to be established within the culture space 25 thus allowing for precise control of the microenvironment of the cultured cell. By controlling the number, size, and configuration of the openings 34, along with volume of the culture space 25, gradients may be established that allow replication of an in vivo biological environment such that growth of cells may be performed in an environment that mimics the in vivo environment. An opening may act as sources of diffusion of substances or molecules in the bulk media contained exterior to the culture space 25 into the culture space 25 and/or may act as sinks of diffusion of substances or molecules generated by the cells within the culture space 25.

As can be appreciated, openings 34 allow for a concentration gradient of a substance to be established within the culture space 25 thus allowing for precise control of the microenvironment of the cultured cell. By controlling the number, size, and configuration of the openings 34, along with the volume, major dimension and minor dimension of the culture space 25, the REEC of the present invention can be configured for multiplexed analysis.

In one embodiment of the REEC of the present invention, the volume of the fluid medium in the space 25 is less than 0.1%, 1%, 5%, 10%, 20%, 30%, 40%, 50%, 60%, 70%, 80%, 90%, or 100% compared to the total volume of the fluid medium in the chamber.

In another embodiment of the REEC of the present invention, the volume of the fluid in the chamber is greater than at least 1, 2, 5, 10, 100, 1,000, 10,000, 100,000, or 1,000,000 times greater than the volume of the fluid medium in the space 25, providing a source or sink for culture substances or test substances.

As can be appreciated, multiple spaces 25 may be created within a chamber defined by multiple opposing surfaces. For example FIG. 9D provides an embodiments utilizing three planar surfaces to define two culture spaces. Each surface may have any variety of configurations of openings to allow for generation of a variety of gradient configurations using any number of substances. In various embodiments, the culture system may include 1, 2, 3, 4, 5, 7, 8, 9, 10 or greater surfaces for create at least 1, 2, 3, 4, 5, 6, 7, 8, 9 or 10 culture spaces with a chamber. As such, where two opposing surfaces define the space 25, the concentration of at least one substance in the fluid medium is at least about 1%, 2%, 5%, 10% or 50% different at one or more points within the space 25 than outside the space, at one or more times. In another embodiment, where more than two opposing surfaces define two or more spaces 25, the concentration of at least one substance in the fluid medium is at least about 1%, 2%, 5%, 10% or 50% different at one more points within one of the spaces than within one or more of the other spaces, or outside the spaces, at one or more times.

As used herein, “substances” include “culture substances,” “test substances,” or “molecules” the names of which are used interchangeably herein, and are defined as a collection of covalently attached atoms having a molecular weight of less than 2 million Dalton and that have a finite (more than 0) solubility. This includes associated or aggregated molecules such as, for example, protein complexes or drug-receptor pairs.

Substances may at times be referred to as culturing substances and may include a variety of various molecules known in the art to be useful for, associated with, or assayed via cell culture. Substances may at times be referred to as molecules and may be nutrients, cellular waste products, toxic compounds, signaling molecules, growth factors, biomolecules, proteins, gases, such as oxygen, nitrogen, carbon dioxide, naturally occurring chemicals or compounds, environmental or industrial chemicals, drugs, pharmaceutical agents, sugars, such as glucose, and any combination thereof. Substances may at times be referred to as test substances may include a variety of various molecules known in the art to be useful for, associated with, or assayed via cell culture.

In various embodiments, a maximum rate of diffusive exchange of the substance is defined by the opening. In some embodiments the maximum rate of diffusive exchange of the substance between space 25 and chamber 26 parallel to the minor dimension is less than about 0.999, 0.99, 0.95, 0.9, 0.8, 0.7, 0.5, 0.1, 0.01, or 0.001 times that of the maximum rate of diffusive exchange of the substance parallel to the major dimension.

As discussed herein, REECs of the present invention may be used with a variety of cell types and sizes. For example, in one embodiment, adherent cells or cells that can be attached to the first surface of the chamber may be used. An illustrative list of eukaryotic cell types that can be used includes primary cells; fibroblasts; motile cells, ciliated cells; cancer cells including cervix, ovary, colorectal breast, prostate, bladder, pancreas, kidney, lung, salivary gland, testis, cecum, liver, colon, mammary gland, vulva, stomach, pleura, bladder, brain, bone, bone marrow, lymph, eye, connective tissue, pituitary gland, muscle, heart, spleen, skin, uterus, endometrium cells, epithelial cells; endothelial cells; blood cells; neural cells; secretory cells including adrenal gland cells; contractile cells including smooth muscle cells and skeletal muscle cells; hepatocytes; adipocytes; lymphocytes; macrophages; T-cells; B-cells; dendritic cells; neurons; chrondrocytes, and stem cells including embryonic, fetal, amniotic, adult and induced pluripotent stem cells. Examples of some cell types listed above include Swiss 3T3, NIH 3T3, MDA-MB-231, MCF-7, HEPG2, CHO, CACO-2, MDCK, B16-F1, B16-F10, HUVEC, PC-12, WI-38, HDF, and SW-13 cell lines. Also included as examples are adherent cells in the American Type Culture Collection's (ATCC) list of tumor cell lines and all the adherent primary cell lines (17 lines) and adherent cell and hybridoma lines (1627) at the ATCC as of the filing of this application.

The cells may be cultured in fluid culture medium, for example an appropriate nutrient medium and incubation conditions that supports growth of cells. Many commercially available media such as Dulbecco's Modified Eagles Medium (DMEM), RPMI 1640, Fisher's, Iscove's, and McCoy's, may be suitable for supporting the growth of the cell cultures. The medium may be supplemented with additional substances such as salts, carbon sources, amino acids, serum and serum components, vitamins, minerals, reducing agents, buffering agents, lipids, nucleosides, antibiotics, attachment factors, and growth factors. Formulations for different types of culture media are described in various reference works available to the skilled artisan (e.g., Methods for Preparation of Media, Supplements and Substrates for Serum Free Animal Cell Cultures, Alan R. Liss, New York (1984); Tissue Culture: Laboratory Procedures, John Wiley & Sons, Chichester, England (1996); Culture of Animal Cells, A Manual of Basic Techniques, 4 th Ed., Wiley-Liss (2000)).

An REEC of the present invention may also incorporate other physical, chemical, or electronic components necessary or desirable for a given objective of the cell culture. For example, it may be connected to other apparatus and instruments for proper operation of the culturing processes, as would be apparent to and understood by a person skilled in the art of bioprocess engineering. In one embodiment, the system may further include a fluid exchange apparatus to exchange or modify the solution in the bulk or within the chamber manually or automatically. For example, the fluid exchange apparatus may be operatively connected with the chamber 26.

In another embodiment a REEC of the present invention is at a uniform temperature.

In another embodiment a REEC of the present invention has no convective flow.

In another embodiment a REEC of the present invention is mechanically isolated, or in a chamber or environment that is mechanically isolated.

In another embodiment a REEC of the present invention a convective flow is introduced to eliminate or reduce one or more gradients for some period of time. Convective flow can for example be introduced by mechanical agitation, fluidic devices, and thermal gradients.

A control system such as a computer or other automation devices (not shown in the figures) may be used to monitor and control the operation of the REEC, and to analyze obtained data. The culturing environment in chamber 26 may be adjusted dynamically based on the information gathered in real time. Media flow and metabolite concentrations can be monitored and controlled. For example, sensors may be connected to the control system using electrodes and may be used to simultaneously measure the concentration of oxygen and the pH value in culture space 25. Further, with multiple sensors, the gradient of a given material in the chamber can be measured. Feedback information may include values of pH, glucose and oxygen concentrations, temperature, osmolarity, shear forces, and the like.

Electrical conductors (not shown) may be embedded in the REEC for connecting sensor and pump electrodes to external electronics and power sources. The conductors may be deposited using standard microelectronics fabrication techniques. For example, the conductors may have a thickness on the order of nanometers. A conductor may run along the surface of a substrate or through the substrate. Conductors may also be covered with inert coatings with non-conducting materials such as aluminum oxide.

As mentioned earlier, computers and computer programs can be used to control the culturing and monitoring of cells and to analyze obtained data. Microprocessors can be incorporated into the REEC or in a separate centralized unit. The computer system can record the measurements from the sensors, analyze the data, and control the culture parameters accordingly. The culture chamber and accessory devices may be monitored and controlled by one or multiple processors and software programs. For example, the system may be programmed to move or adjust surfaces defining the culture space 25 to change the volume or shape of the space in response to data gathered by sensors or other programmed fashion, e.g., at specific time intervals.

As can be appreciated, since the REEC confines growth of a cell, it is easier to control the characteristics of the cell's environment. For example, neighbouring cells of a target cell can be selectively deposited. The neighbouring cells may be of one or more particular cell types different from the target cell type. The concentration gradients of certain components of the culturing medium around the target cell can be precisely controlled. The target cell can be monitored continuously and its growth or division history can be tracked and recorded. Since the characteristics of the first 22 and second surfaces 30 can be pre-selected, it is possible to prevent exposure of cells to a surface with uncontrolled or undesirable characteristics.

In culture of a monolayer of cells using the REEC, it is possible to remove a particular cell from the monolayer of cells without significantly disturbing the neighbouring cells. As will be understood by persons skilled in the art, removal of a particular cell may be desirable, for example, when the particular cell is observed to meet one or more criteria which may be related to karyotype, morphology and cell size.

In one embodiment, one or more cells can be cultured while the movement of the cell(s) is restricted such that each cell is in continuous contact with two opposing surfaces. However, each cell, or the cells as a whole, is mobile between the surfaces, i.e. movable in a direction generally parallel to the surfaces. Conveniently, the cell(s) can be imaged during culturing via techniques generally known in the art. For example, a non-confocal imaging device may be used, such as a bright field imaging device or a fluorescent imaging device including a differential interference contrast (DIC) imaging device. High resolution images can be obtained without using a confocal microscope because the cell(s) can be confined within the proximity of the focal plane of a non-confocal imaging device. Thus, cell(s) can be monitored, including imaged, over a long period of time (up to many days) without being significantly adversely affected. The imaging is not restricted to non-confocal devices, but can be performed with a confocal imaging system or any imaging system that can be used to image cells in culture.

In operation, a number of desired cells are deposited in chamber 26. The cells are then confined between opposing surfaces 22 and 30. In various embodiments, one or more other cells (e.g., cells of a different cell type) may be disposed around the initially deposited cells. In such cases, the other cells also help to confine a target cell. The cells may be specifically positioned in chamber 26 manually, or they may position themselves through self-organization. As can be appreciated, two adjacent cells can mutually constrain each other when they are compressed towards each other.

Cells may be seeded into chamber 26 be any method known in the art. For example, cells may be deposited with a micropipette from the top of the chamber with cover 28 removed. Cover 28 may be lowered into position slowly so as not to disturb the cells. Once delivered, cells may self-organize or self-assemble in the culture chamber 26, which can be facilitated by the configuration of the openings 34 and surfaces in contact with the culture chamber which may have pre-selected suitable characteristics such as appropriate affinities to specific cells.

In various aspects, the REEC of the present invention may be used in various applications. In one aspect, the REEC may be used to model tumors as described further in the Examples.

In another aspect, the REEC may be used to model ischemia. Ischemia occurs both under normal and pathological or disease conditions. Pathological or disease examples of ischemia include heart attack, stroke, renal ischemia and diabetes. REECs in combination with the appropriate cell type, or combination of cell types, can be used to model these processes by introducing complex and realistic barriers to diffusive exchange. The system of the present invention allows cells to be exposed to gradients and non-uniform concentrations of molecular constituents. This collection of different models can then be used for biomedical research, as well as drug development and testing.

In another aspect, the present invention provides a method for assaying healing or recovery responses of cells. In various embodiments, cultured cells may be monitored before and after specific growth conditions or exposure to agents. For example, cells may be culture using the culture system according to the method described herein, wherein the second surface is removed and the cells are further cultured without the second surface being present. The growth of the cells before and after removal of the second surfaces may be compared to assess various parameters of the culture conditions. This approach is useful to study healing or post insult recovery responses. For example, cells grown in an REEC will sometimes die or develop various stressed responses to the environment. Once this situation develops, the cover may be removed, and the recovery of the cells to more fully exchanging environments can be studied. Alternately, an REEC may be modified by introducing a cover with a different shape or configuration of openings.

In another aspect, the present invention provides a method for assaying interactions of two or more soluble components or components at two or more concentrations. A system of the present invention may be configured such that it has two or more openings, in contact with two or more bulk solutions, where the bulk solutions have different compositions (FIG. 9A). For example, the solutions may have two or more different drugs, an agonist in one and an antagonist in the other, high concentration of growth factor in one and low concentration in the other, and so on. This configuration results in gradients of multiple components mixing in complex ways, depending on the specific geometry of the openings.

In another aspect, the present invention provides a method for performing a titration assay utilizing the system of the invention. The method includes a) providing a chamber having a first surface in opposition to a second surface defining a space in the chamber, the first surface and second surface being spaced at a distance to allow culture of one or more cells between the first surface and the second surface; b) introducing one or more cells to the space in the chamber, wherein the space comprises a fluid culture medium; c) providing an agent to the space and allowing a concentration gradient of the agent within the space to be established via an opening in the second surface; and d) detecting a response of the one or more cells to the agent at time intervals as the concentration gradient is established, thereby performing a titration assay. By introducing a drug or some other agent into the bulk, a gradient of that molecule will develop in the culture space over some period of time. During that time dose dependent responses can be quantified along the various axes of diffusion (depending on the shape of the opening).

In another aspect, the present invention provides a method for performing a cell migration assay utilizing the system of the invention. The method includes a) providing a chamber having a first surface in opposition to a second surface defining a space in the chamber, the first surface and second surface being spaced at a distance to allow to be cultured between the first surface and the second surface; b) introducing one or more cells to the space in the chamber, wherein the space comprises a fluid culture medium; c) providing to the cell a substance, wherein the substance is provided through an opening in the second surface, wherein a concentration gradient of the substance within the space is established; and d) detecting an increase or decrease in migration of the one or more cells along the concentration gradient, thereby performing a cell migration assay. In this manner, the system of the present invention may be used to study and/or quantify the dynamics of cell movements in complex gradients. Agents that increase or decrease cell migration can also be added to the bulk at various times, resulting in gradients along which cells may increase or decrease their migration.

In another aspect, the present invention provides a method of generating a tissue utilizing the culture system of the invention. The method includes a) providing one or more cells of a type appropriate for a specific tissue type; b) culturing the cells using the system described herein; and c) harvesting the resultant tissue, thereby generating a tissue. The differentiation and growth of the cells may controlled via introduction and selection of the substance in combination with configuration of the system. The various embodiments, the tissue may be a one, two or three-dimensional tissue, and incorporate one, or a variety of specialized cell types. The shapes of tissues and organs are determined in part by gradients of soluble molecules, or gradients of insoluble molecules that arise as a consequence of either nonuniform exposure to soluble molecular constituents. Thus the ability to control the form of gradients permits the control of how collections of cells, a single cell type or two or more cell types in a co-culture, arrange themselves spatially, change or modify their internal structure and composition, or change or modify the extracellular environment, in order to form functionally important units, elements, aggregates, associations, organs, tissues and structures.

In another aspect, the present invention provides a kit for performing cell culture. In various embodiments, the kit includes a culture system of the present invention including a chamber for receiving a fluid culture medium; a first surface and a second surface defining a space in the chamber, wherein the second surface includes one or more openings for passage of a substance into or out of the culture space. The kit may optionally further include cells, culture media, reagents for detecting a cellular or biological activity, instructions for using the kit in accordance with any of the described methods, and tools for customizing the culture system. The kit may be configured in a multiwell format for high throughput automation.

The following examples are provided to further illustrate the advantages and features of the present invention, but are not intended to limit the scope of the invention. While they are typical of those that might be used, other procedures, methodologies, or techniques known to those skilled in the art may alternatively be used.

### Example 1

### Culture of Human Adenocarcinoma Cells

This example illustrates culture of MDA-MD-231 cells (a human adenocarcinoma cell line) using an REEC culture system according to one embodiment of the present invention.

With reference to FIG. 2, 100 μm glass spheres or other small spacers 32 were attached to 20 mm diameter glass coverslips 28 at three or more well-distributed points near the edge. The spacer can also have other forms, such as a single continuous thin film cut to the shape of a ring that lies near the edge of the coverslip. The spacer can also be engineered into the coverslip using for example etching methods. These coverslips were then machined to produce an opening 34, such as a round 0.2 mm hole or a 1 cm×0.5 mm bar (FIGS. 2B and 2C), near the center. Cells were then seeded into a 12 well cell culture plate, where the diameter of each well is 22 mm and grown to a desired confluency in culture medium. The cells used were MDA-MD-231 cells, although any cell type can be used. The coverslips were then placed into the wells, with the spheres positioned such that they serve as spacers—creating a culture space 25 that is effectively 20 mm in diameter and 100 μm thick. The coverslips 28 were held down by a thin plastic loop that presses against the cover when engaged, or some other means, to prevent the coverslip 28 from being displaced by movement of the dish, convective flows or air/gas bubbles forming inside the chamber.

The cells were incubated for some desired period of time during which time the dynamics of cells in response to the forming gradients were recorded optically. The growth of the cells in this type of experiment is shown schematically in FIG. 2D. The coverslips were removed, and the cells in the wells immediately fixed to preserve the sample for later analysis with antibodies or other reagents.

One useful variation was to place a 20 mm coverslip in the bottom of the chamber 26 or well prior to plating cells. The lower coverslip can then be recovered at the end for higher performance microscopy than would be possible through the plastic plate (or one can use a glass bottom dish).

The cells in the wells or on a coverslip were stained with antibodies for actin and fibronectin, and the nuclear stain DAPI. The stained samples were visualized by fluorescence microscopy (data not shown). Cell viability was shown to decrease as a function of distance from the opening (FIG. 6).

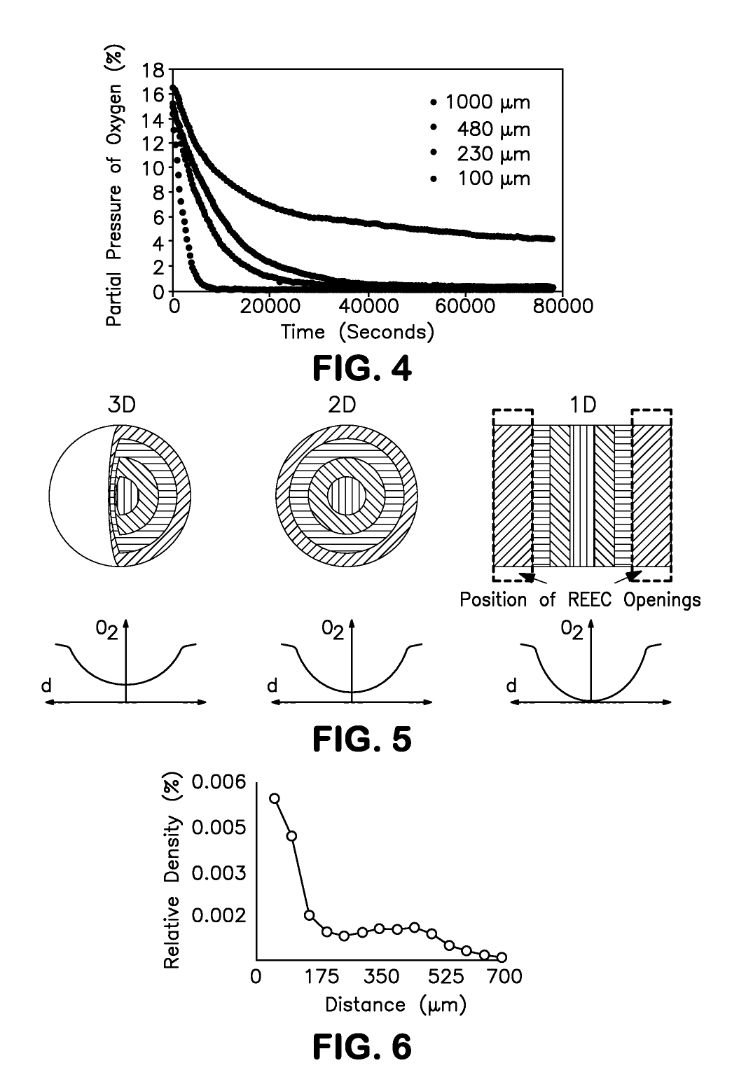

To quantify oxygen dynamics in the REEC geometry cells were cultured as described above using a system having a culture space of 15 mm diameter utilitizing spacers of 100 μm. The cover included only exchange at the edges. The system was configured as a 24-well plate. The partial pressure of oxygen at the center of the culture space was detected over a time course as shown in FIG. 4 which shows that partial pressure of oxygen at the center of the culture space falls to ˜0 in <2 hours (values are averages of three measurements each).

### Example 2

### Use of System to Model Tumors

This example illustrates use of an REEC culture system according to embodiments of the present invention to model tumors.

The culture system of the present invention may be used to model tumors in one, two or three dimensions. The present invention may be used to produce a tumor model, for research and drug screening applications, in which the diffusion-limited exchange of extracellular molecules is more reliably and or cost effectively captured than with current approaches.

A tumor model is composed of a REEC where the second surface has two parallel bar shaped openings machined in it. These openings may be from about 0.5 to 1 mm apart, and thereby form a pair of gradients that meet at the center point between the openings. This amounts to reducing a spheroid to a 1-dimensional structure (FIG. 5).

FIG. 5 is a schematic showing dimensional reduction from a three dimensional spheroid to one dimensional geometry. The model utilizes two parallel bar shaped openings machined in parallel, separated by about 500 μm (dashed lines). When cells are grown in the culture system, the cells near the center line are most exchange restricted and are geometrically equivalent to the center to a spheroid One notable difference between the spheroid and the lower dimensional structures is that, as the dimension decreases, the steepness of the gradient will increase. But this can be accounted for by changing the distance between the openings.

In the one dimensional tumor model the distance between two line openings is the equivalent of the diameter of a three dimensional spheroid, thus the diameter is an easily accessible experimental variable. The model also captures the overlapping gradients equivalent to the center of a spheroid. Further, the analysis of results from the one dimensional system are greatly facilitated by being able to simply average observations along the axis of the chamber.

This model was tested using MDA-MB-231 cells grown in a culture system having having parallel bar shaped openings (data not shown). Cells and fibronectin were observed to organize relative to the openings.

The system may also be used as a two-dimensional tumor model. A system including a cover designed in the configuration depicted in FIG. 7G can be used to generate a two dimensional tumor model. The radii of curvature of the two arcs of the openings can be chosen to correspond to that of spheroidal tumors in vivo. The diffusive exchange gradients set up by such a configuration will overlap, resulting in a two-dimensional model for the tumor environment. This tumor model allows simulation of a spheroid tumor in two dimensions, effectively growing a cross section of a spheroid. Experimental manipulation of the cells within the tumor model is simplified. Further, the experimental system is more accessible to optical microscopy and data analysis.

The system may also be used as a tumor model termed an “inside-out” tumor model. A system including cover having a single circular opening as depicted in the embodiments shown in FIG. 7C can be used as an “inside out” tumor model. In this instance, the diffusive exchange gradients are radially symmetric around the center of the hole. In the case in which the volume above the cover contains a nutrient rich medium, the cells growing directly under the opening are exposed to a high concentration of nutrients, and those at further distances from the opening are exposed to lower concentrations of nutrients. The cells at distances from the center of the opening that correspond to a spheroid radius, will function similar to cells at the center of a spheroid, and those under the opening will function more like cells on the exterior surface of a spheroid.

This collection of different models can further be used for biomedical research; drug development and testing; and toxicity testing and screening.

### Example 3

### Use of System to Study Extracellular Matrix

This example illustrates use of a REEC culture system to study the biology of extracellular matrix (ECM).

It is hypothesized that diffusive gradients that arise from the exchange of metabolites and small molecules between the capillaries and the tissues are necessary to produce structural anisotropy in the ECM. Implicit in this hypothesis is that at least one molecular constituent of the ECM in the vicinity of capillaries, is under some circumstances aligned along the axis of diffusion between the blood and the tissues. This mechanism effectively means that information in diffusive gradients is being encoded into spatial relationships of molecules in the ECM. The spatial organization in the matrix, while not static, will be temporally more stable than a diffusive gradient. For example, if blood supply to a tissue is interrupted the diffusive gradients between the tissues and the capillaries will quickly vanish—while the matrix anisotropy will persist for at least some period of time. The system of the present invention will be used to research ECM.

REECS will be used to demonstrate the formation of anisotropic extracellular along the axis of diffusive exchange.

Fibroblasts are grown in REECs for 5-7 days, during which time they deposit a natural extracellular matrix.

The second surface of the REEC chamber is removed. Natural cell-derived matrix (CDM) produced by cells in culture will be isolated. Preparation of CDM involves culturing a matrix producing cell, like a fibroblast, for 7 days. The cells are then removed by a sequential treatment of 0.05% Triton X-100, 50 mM NH₄OH and DNAse I. The remaining matrix is structurally and functionally intact.

Anisotropic constituents of the ECM will be quantified using immunofluorescence microscopy. The molecular constituents examined will include, fibronectin, collagen I, tenascin-C, collagen IV, versacan and laminin.

These experiments will then be repeated for varying steepness of the diffusive gradients. The steepness of gradients can be controlled by the thickness of the spacers used. Thus the above experiments will be performed with 100, 200, and 400 μm spacers. If the diffusive gradient is required for matrix anisotropy, then the extent of alignment should depends on the steepness of the gradient.

CDM from ECM produced in REECs will be used to determine the role that anisotropic extracellular matrix plays in directional cell movement. The alignment of ECM relative to the axis of diffusive exchange raises the question of what role the matrix might play in directional movement of cells relative to the gradients of diffusive molecules. Addressing this question requires being able to uncouple the diffusive gradient from the aligned ECM. The general approach is to produce a CDM from fibroblasts or MDA-MB-231 cells grown with or without a culture system of the present invention, and then examine how cells migrate on these surfaces in the absence of any diffusive gradients.

### Example 4

### Use of System to Study Breast Cancer

This example illustrates use of a REEC culture system to study the biology of breast cancer.

The hypothesis addressed here is that diffusive exchange of small molecules and metabolites plays an important role in breast cancer physiology. Specifically it is hypothesized that gradients along which diffusion occur modifies the local matrix microenvironment, which in turn changes influences the growth and spatial organization of resident cells.

REECS are used to determine how restricted diffusion alters the biochemistry and morphology of a cell-derived extracellular matrix from breast fibroblast or breast cancer cells. Using a combination of an approach for producing a functional cell-derived extracellular matrix as described in Example 3 and a cell culture system of the present invention, the effect that diffusive gradients have on the molecular composition and spatial organization of ECM will be established.

REECS are used to determine how restricted diffusive exchange alters the micromechanics of a cell-derived extracellular matrix from breast fibroblast or breast cancer cells. Using the approach from example 3 to produce extracellular matrix along diffusive axes, the effect of this type of non-uniform environment on the micromechanical properties of the ECM by atomic force microscopy determine.

REECS are used to determine how extracellular matrix modified by diffusive gradients affects the morphology and growth of breast cancer cells. Using the approach from aim 1 to produce extracellular matrix along diffusive axes, how this matrix modifies cells shape and supports tumor cell growth will be tested.

The approach will be to produce cell-derived extracellular matrices under conditions where the diffusive exchange is limited, such that a gradient of conditions is established—similar to the conditions that occur as a function of distance from a blood vessel or the outside of a spheroid. That matrix will then be characterized biochemically, morphologically and mechanically. Further, the ability of this cell-derived matrix to modify the organization and growth of tumor cells will be quantified. Additional experiments will be performed to examine matrix remodeling, by adding or removing the restricted diffusion at different times during matrix biosynthesis.

### Example 5

### Modeling of Variations in Capillary Density

REECS can be used to model a tissue. For example, a single round opening that is 200 μm in diameter that connects the space containing cells with the bulk medium serves as a model for a capillary. The diffusion from the opening gives rise to radial gradients, similar to a cross section through a Krogh cylinder. Such tissue models can be manipulated by placing round openings at different densities on one of the surface, which models multiple capillaries. The densities of openings in the REECs can then be pre-defined based on experimental measurements, or based on computational or mathematical models. The densities can also be selected such that there is either excess or insufficient diffusive exchange for some physiological or disease process.

### Example 6

### Screening, Testing or Characterizing Therapeutic Agents

Systemic therapeutic agents are delivered via the circulatory system, and enter the tissues via diffusion. This process produces a concentration profile that is both temporally and spatially highly inhomogeneous. The pharmacokinetics receives a great deal of attention, but the question of spatial distribution is more difficult to deal with—especially since the some of the inhomogeneities occur on a length scale of ˜100 um. REECs can be used to characterize or quantify the effects of these inhomogeneties in cell based assays, by taking into account spatial variations in concentrations of small molecules and cell physiology. For example, it will no longer be a question of an LD₅₀ for all cells in a culture, but an LD₅₀ as function of distance from the nearest capillary. This provides a means to identify refractory classes of cells that in some cases reduce the efficacy of a treatment. Further, the spatial distribution of a drug is closely tied to its schedule of administration. Thus, an understanding of the relationship between efficacy and spatial distribution could provide new insights to dosing schedules.

### Example 7

### Screening, Testing or Characterizing Toxic Therapeutic Agents

Systemic toxic agents are delivered via the circulatory system, and enter the tissues via diffusion. This process produces a concentration profile that is both temporally and spatially highly inhomogeneous. REECs can be used to characterize or quantify the effects of these inhomogeneties in cell based assays, by taking into account spatial variations in concentrations of small molecules and cell physiology. For example, it will no longer be a question of an LD₅₀ for all cells in a culture, but an LD₅₀ as function of distance from the nearest capillary. This provides a means to identify classes of cells more or less sensitive to the toxin. Further, the spatial distribution of a toxin is closely tied to its schedule of administration. Thus, an understanding of the relationship between efficacy and spatial distribution could provide new insights to exposure limits and safety.

### Example 8

### Prolonged Culture with Stable Gradients

The stability of the gradients formed in REECs depends in part on the relative volume the fluid culture medium in the space 25 and total volume of the fluid culture medium in the chamber. One way to achieve more stable gradients for longer periods of time is to make the volume of the medium outside space 25 large relative to the volume in the space 25. For example, a REEC with a space 25 with a 30 μl volume (for example in a well of a 12-well plate), a volume of 4 ml of medium in the well would be a better sink or source than a 1 ml volume. Alternatively the wells can also be connected to fluidic devices that continuously or periodically exchange the medium in the well. This replenishes substances that might be consumed in the space, or removes substances that might be produced in the space.

Although the invention has been described with reference to the above example, it will be understood that modifications and variations are encompassed within the spirit and scope of the invention. Accordingly, the invention is limited only by the following claims.

What is claimed is:

1. A cell culture system, comprising:
a) a chamber for receiving a fluid culture medium;
b) a first surface and one or more surfaces in opposition thereby defining one or more spaces in the chamber; and
c) one or more cells within the one or more spaces;
wherein the one or more surfaces comprise one or more openings allowing for passage of a substance into or out of the one or more spaces.

2. The cell culture system of claim 1, wherein the cells are growing on or by other means attached to the internal side of the one or more surfaces.

3. The cell culture system of claim 1, wherein the cells are free floating.

4. The cell culture system of claim 1, wherein the cells are confined by a permeable barrier.

5. The cell culture system of claim 1, wherein one or more of the surfaces are substantially planar.

6. The cell culture system of claim 1, wherein one or more of the surfaces are transparent.

7. The cell culture system of claim 1, wherein one or more of the surfaces are opaque.

8. The cell culture system of claim 1, wherein one or more of the surfaces comprise a cell culture compatible material.

9. The cell culture system of claim 1, wherein each space comprises a major dimension and a minor dimension, the minor dimension being defined by the distance between two opposing surfaces that define said space, wherein the minor dimension is smaller than the major dimension, and wherein the major dimension of at least one of the spaces is at least about 2, 3, 5, 10, 20, 50 or 100 times larger than the minor dimension.

10. The cell culture system of claim 9, wherein the minor dimension varies at different positions along the major dimension in one or more of the spaces.

11. The cell culture system of claim 9, wherein the minor dimension varies at different distances from an opening in one or more of the spaces.

12. The cell culture system of claim 1, wherein two or more of the surfaces are mounted at a fixed separation distance or distances.

13. The cell culture system of claim 1, wherein two or more of the surfaces are separated by one or more spacers to define the space.

14. The cell culture system of claim 1, wherein at least one of the surfaces is moveable for adjusting the size of the space.

15. The cell culture system of claim 1, wherein at least one of the surfaces is movable via pre-programming to adjust the size of the space as a function of time.

16. The cell culture system of claim 1, wherein at least one surface is programmed to move in response to one or more sensors that detect one or more parameters of the culture system or contents thereof.

17. The cell culture system of claim 1, wherein at least one surface is removable.

18. The cell culture system of claim 9, wherein the minor dimension of at least one of the spaces is less than about 5, 2, 1, 0.5, 0.1, or 0.05 mm.

19. The cell culture system of claim 1, wherein two opposing surfaces define the space, and wherein the concentration of at least one substance in the fluid medium is at least about 1%, 2%, 5%, 10% or 50% different at one or more points within the space than outside the space, at one or more times.

20. The cell culture system of claim 1, wherein more than two opposing surfaces define two or more spaces, and wherein the concentration of at least one substance in the fluid medium is at least about 1%, 2%, 5%, 10% or 50% different than at one or more points within one of the spaces than within one or more other spaces or outside the spaces, at one or more times.

21. The cell culture system of claim 9, wherein the opening has an area parallel to the major dimension of less than about 90, 80, 70, 60, 50, 40, 30, 20, 10, 5, or 1% of the area of the opposing surface defining the space.

22. The cell culture system of claim 1, wherein one or more surfaces comprise a plurality of openings.

23. The cell culture system of claim 1, further comprising a plurality of chambers separated by dividers.

24. The cell culture system of claim 1, further comprising a plurality of chambers configured as a multi-well plate.

25. The cell culture system of claim 1, further comprising an imaging device.

26. The cell culture system of claim 1, further comprising one or more sensors for monitoring the fluid culture medium or the cells.

27. The cell culture system of claim 26, where the one or more sensors is positioned in one or more of the spaces, or outside one or more of the spaces.

28. A method of cell culture comprising:
a) providing a chamber having a first surface in opposition to one or more second surfaces defining one or more spaces in the chamber, the first surface and one or more second surfaces being spaced at a distance to allow one or more cells to adhere to the first surface;
b) introducing one or more cells to the one or more spaces in the chamber, wherein the spaces comprise a fluid culture medium;
c) providing to the cell a substance, wherein the substance is provided through an opening in the one or more second surfaces, wherein a concentration gradient of the substance within the one or more spaces is established, thereby culturing the cells.

29. The method of cell culture of claim 28, wherein the cells are growing on or by other means attached to the internal side of the one or more surfaces.

30. The method of cell culture of claim 28, wherein the cells are free floating.

31. The method of cell culture of claim 28, wherein the cells are confined by a permeable barrier.

32. The method of cell culture of claim 28, wherein one or more of the surfaces are substantially planar.

33. The method of cell culture of claim 28, wherein one or more of the surfaces are transparent.

34. The method of cell culture of claim 28, wherein one or more of the surfaces are opaque.

35. The method of cell culture of claim 28, wherein one or more of the surfaces comprise cell culture compatible materials.

36. The method of cell culture of claim 28, wherein each space comprises a major dimension and a minor dimension, the minor dimension being defined by the distance between the opposing surfaces that define said space, wherein the minor dimension is smaller than the major dimension, and wherein the major dimension of at least one of the spaces is at least about 2, 3, 5, 10, 20, 50 or 100 times larger than the minor dimension.

37. The method of cell culture of claim 36, wherein the minor dimension varies at different positions along the major dimension in one or more of the spaces.

38. The method of cell culture of claim 36, wherein the minor dimension varies at different distances from an opening in one or more of the spaces.

39. The method of cell culture of claim 28, wherein two or more of the surfaces are mounted at a fixed separation distance or distances.

40. The method of cell culture of claim 28, wherein two or more of the surfaces are separated by one or more spacers to define the space.

41. The method of cell culture of claim 28, wherein at least one of the surfaces is moveable for adjusting the size of the space.

42. The method of cell culture of claim 28, wherein at least one of the surfaces is movable via pre-programming to adjust the size of the space as a function of time.

43. The method of cell culture of claim 28, wherein at least one surface is programmed to move in response to one or more sensors that detect one or more parameters of the culture system or contents thereof.

44. The method of cell culture of claim 28, wherein at least one surface is removable.

45. The method of cell culture of claim 36, wherein the minor dimension of at least one of the spaces is less than about 5, 2, 1, 0.5, 0.1, or 0.05 mm.

46. The method of cell culture of claim 28, wherein two opposing surfaces define the space, and wherein the concentration of at least one substance in the fluid medium is at least about 1%, 2%, 5%, 10% or 50% different at one more points within the space than outside the space, at one or more times.

47. The method of cell culture of claim 28, wherein more than two opposing surfaces define two or more spaces, and wherein the concentration of at least one substance in the fluid medium is at least about 1%, 2%, 5%, 10% or 50% different at one or more points within one of the spaces than within one or more of the other spaces or outside the spaces, at one or more times.

48. The method of cell culture of claim 36, wherein the opening has an area parallel to the major dimension of less than about 90, 80, 70, 60, 50, 40, 30, 20, 10, 5, or 1% of the area of the opposing surface defining the space.

49. The method of cell culture of claim 28, wherein the one or more surfaces comprise a plurality of openings.

50. The method of cell culture of claim 28, further comprising a plurality of chambers separated by dividers.

51. The method of cell culture of claim 28, further comprising a plurality of chambers configured as a multi-well plate.

52. The method of cell culture of claim 28, further comprising providing an imaging device.

53. The method of cell culture of claim 28, comprising monitoring the fluid culture medium or the cells with one or more sensors.

54. The method of cell culture of claim 53, where the one or more sensors is positioned in one or more of the spaces, or outside one or more of the spaces.

55. The method of cell culture of claim 28, further comprising continuously changing the medium outside the one or more spaces.

56. The method of cell culture of claim 55, wherein the cell culture medium outside the one or more spaces is exchanged at least about every 8 hours, 4 hours, 2 hours or 1 hour.

57. The method of claim 28, wherein (c) further comprises providing at least one additional substance through the opening, wherein at least one additional concentration gradient of the at least one additional substance within the space is established.

58. The method of claim 28, wherein (c) further comprises providing additional substances through at least one additional opening, wherein additional concentration gradients of the additional substances within the space is established.

59. The method of claim 28, wherein the one or more surfaces are introduced into the chamber at one or more times after the culture of cells has been initiated.

60. The method of claim 28, further comprising obtaining an image of the one or more cells.

61. The method of claim 60, wherein one or more of the second surfaces and the culture medium are removed before obtaining the image.

62. The method of claim 28, further comprising:
d) removing the one or more surfaces;
e) culturing the cells without the one or more second surfaces present; and
f) comparing growth of the cells after culture without the one or more second surfaces as to growth before the one or more second surfaces is removed.

63. The method of claim 62, wherein one or more surfaces is reintroduced, reforming a chamber space that is the same or different than the original chamber space.

64. The method of claim 28, wherein the a cell migration assay is performed, the method further comprising detecting an increase or decrease in migration of the one or more cells along the concentration gradient, thereby performing a cell migration assay.

65. The method of claim 28, wherein a titration assay is performed, the method further comprising detecting a response of the one or more cells to the substance at time intervals as the concentration gradient is established, thereby performing a titration assay.

66. The method of claim 28, wherein tissue generation is performed, wherein the one or more cells are of a type appropriate for a specific tissue type and differentiation and growth of the cells is controlled during culture via introduction and selection of the substance.

67. The method of claim 66, further comprising harvesting the cultured cells.

68. The method of claim 66, wherein the tissue is a one, two or three-dimensional tissue.

69. The method of claim 66, wherein the tissue is a model for a tumor or ischemia.
